(19) United States
(12) Patent Application Publication
(10) Pub. No.: US 2026/0340822 A1
(43) Pub. Date: Oct. 29, 2026

(54) DETECTING RESIDUAL LENS FRAGMENTS DURING AN OPHTHALMIC SURGICAL PROCEDURE

(71) Applicant: REGENTS OF THE UNIVERSITY OF MICHIGAN, Ann Arbor, MI (US)

(72) Inventors: Nambi Nallasamy, Ann Arbor, MI (US); Binh Duong Giap, Ann Arbor, MI (US)

(21) Appl. No.: 19/658,018

(22) Filed: Apr. 24, 2026

Related U.S. Application Data
(60) Provisional application No. 63/794,750, filed on Apr. 25, 2025.

(51) Int. Cl. G06T 7/00; G16H 50/20

(52) U.S. Cl. CPC G06T 7/0012; G16H 50/20; G06T 2207/20081; G06T 2207/20084; G06T 2207/30041

(57) ABSTRACT

System and methods for detecting a residual lens fragment using machine learning are provided. An example system may obtain surgical image data comprising a plurality of images capturing an ophthalmic surgical procedure of a subject. The system may provide the surgical image data to a machine learning model trained to detect at least one residual lens fragment within an eye of the subject during the ophthalmic surgical procedure based upon analyzing the surgical image data. The system may determine whether the at least one residual lens fragment meets at least one detection criteria, and based upon determining the at least one residual lens fragment meets the at least one detection criteria, generate output data associated with detecting the at least one residual lens fragment in the eye of the subject. The system may transmit the output data to a computing device.

### CROSS-REFERENCE TO RELATED APPLICATIONS

This application claims the benefit of U.S. Application No. 63/794,750, filed Apr. 25, 2025, and entitled “Detecting Residual Lens Fragments During an Ophthalmic Surgical Procedure”, which is incorporated herein by reference in its entirety.

### GOVERNMENT LICENSE RIGHTS

This invention was made with government support under TW012027, and EY022299 awarded by the National Institutes of Health. The government has certain rights in the invention.

### FIELD OF THE DISCLOSURE

The present disclosure generally relates to systems and methods for analyzing surgical images, and more particularly, to systems and methods for using machine learning to detect residual lens fragments in the eye of a subject during a surgical procedure.

### BACKGROUND

Cataract surgery remains the only definitive treatment for visually significant cataracts, which are a major cause of preventable blindness worldwide. Part of the cataract surgery involves the emulsification of the native, crystalline lens material using an ultrasound device to facilitate lens material removal through small corneal incisions. During this phacoemulsification process, small fragments of lens material, referred to as residual lens fragments, may be created within the eye of the surgical subject. The residual lens fragments can become trapped within incisions, the iridocorneal angle, posterior to the iris, or other portions of the eye, putting the surgical subject at risk for complications such as cystoid macular edema, persistent anterior uveitis, corneal edema, and glaucoma, and necessitating further surgical intervention to remove the residual lens fragments.

Conventional lens fragment detection techniques generally include visually identifying lens fragments within the eye of the patient during the surgical procedure. Unfortunately, due to the transparent and/or brunescent appearance of lens material, even expert surgeons assisted by visual magnification tools such as an operating microscopes may be unable to detect the presence of the lens fragments during the cataract procedure. The inability to detect residual lens fragments may be compounded by the diversity of patient eye anatomies and colors which can further obscure the presence of residual fragments.

Given these challenges, there are opportunities for systems and methods that provide improved detection and tracking of residual lens fragments during ophthalmic surgery procedures to reduce the risk of postoperative complications, improve patient outcomes, decrease the need for additional surgical interventions, and otherwise avoid the shortcomings and inconsistencies of the present techniques.

### BRIEF SUMMARY

In one aspect, a computer-implemented method for detecting a residual lens fragment (RLF) using machine learning may include: (i) obtaining, by one or more processors, surgical image data comprising a plurality of images capturing an ophthalmic surgical procedure of a subject; (ii) providing, by the one or more processors, the surgical image data to a machine learning model trained to detect at least one RLF within an eye of the subject during the ophthalmic surgical procedure based upon analyzing the surgical image data; (iii) determining, by the one or more processors, whether the at least one RLF meets at least one detection criteria; (iv) based upon determining the at least one RLF meets the at least one detection criteria, generating, by the one or more processors, output data associated with detecting the at least one RLF in the eye of the subject; and (v) transmitting, by the one or more processors, the output data to a computing device. The computer-implemented method may include additional, less, or alternate functionality or actions, including those discussed elsewhere herein.

In another aspect, a system for detecting a residual lens fragment (RLF) using machine learning may include: one or more processors; and one or more non-transitory memories storing processor-executable instructions that, when executed by the one or more processors, cause the system to: (i) obtain surgical image data comprising a plurality of images capturing an ophthalmic surgical procedure of a subject; (ii) provide the surgical image data to a machine learning model trained to detect at least one RLF within an eye of the subject during the ophthalmic surgical procedure based upon analyzing the surgical image data; (iii) determine whether the at least one RLF meets at least one detection criteria; (iv) based upon determining the at least one RLF meets the at least one detection criteria, generate output data associated with detecting the at least one RLF in the eye of the subject; and (v) transmit the output data to a computing device. The system may include additional, less, or alternate functionality, including that discussed elsewhere herein.

In another aspect, a non-transitory computer readable storage medium storing executable instructions that, when executed by a processor, cause the one or more processors to at least: (i) obtain surgical image data comprising a plurality of images capturing an ophthalmic surgical procedure of a subject; (ii) provide the surgical image data to a machine learning model trained to detect at least one residual lens fragment (RLF) within an eye of the subject during the ophthalmic surgical procedure based upon analyzing the surgical image data; (iii) determine whether the at least one RLF meets at least one detection criteria; (iv) based upon determining the at least one RLF meets the at least one detection criteria, generate output data associated with detecting the at least one RLF in the eye of the subject; and (v) transmit the output data to a computing device. The instructions may direct additional, less, or alternate functionality, including that discussed elsewhere herein.

### BRIEF DESCRIPTION OF THE DRAWINGS

The figures described below depict various aspects of the system and methods disclosed therein. It should be understood that each figure depicts one embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present aspects are not limited to the precise arrangements and instrumentalities shown, wherein:

FIG. 1 depicts a block diagram of an example computing environment in which methods and system for detecting a residual lens fragment using machine learning are implemented, according to embodiments;

FIG. 2 depicts a combined block and logic diagram for training a machine learning model, according to embodiments;

FIG. 3A depicts an example system for detecting a residual lens fragment using machine learning, according to embodiments;

FIG. 3B depicts an example first image of a surgical video capturing an ophthalmic surgical procedure of a subject, according to embodiments;

FIG. 3C depicts an example second image of a surgical video including a semantically segmented residual lens fragment, according to embodiments; and

FIG. 4 an example block flow diagram depicting a computer-implemented method for detecting a residual lens fragment using machine learning, according to embodiments.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

### Overview

The disclosed systems and methods provide techniques for detecting a residual lens fragment (RLF) during ophthalmic surgical procedures using machine learning. The disclosed techniques employ a trained machine learning model to analyze surgical image data comprising images capturing an ophthalmic surgical procedure (e.g., cataract surgery) of a subject to identify one or more RLFs within the eye of a subject. The disclosed techniques determine whether a detected RLF meets at least one detection criteria. Based upon the RLF meeting a detection criteria, the disclosed techniques generate output data associated with the detected RLFs, and transmit the output data to a computing device, such as a surgical computer or display associated with the surgical staff performing the surgical procedure. Thus, the disclosed systems and methods provide streamlined and enhanced techniques for detecting RLFs during surgical procedures.

In some embodiments, the surgical image data comprises a sequence of images (e.g., video frames) captured over time during the ophthalmic surgical procedure. The machine learning model may analyze temporal relationships between the sequence of images to detect and track one or more RLFs across multiple frames.

In such embodiments, the model may determine temporal consistency, motion, persistence, or trajectory of the RLF across frames to improve detection accuracy and reduce false positives. For example, an object may be identified as an RLF based on its consistent presence across a plurality of consecutive frames, its motion relative to anatomical structures, or its behavior across different surgical phases.

The model may include temporal architectures such as recurrent neural network (RNNs), long short-term memory (LSTM) networks, convolutional LSTM (ConvLSTM), 3D convolutional neural networks (3D CNNs), Inflated 3D (i3D) networks, or video transformers.

In some embodiments, the surgical image data may be preprocessed prior to analysis. Preprocessing may include cropping a region of interest (ROI) corresponding to the eye or a portion thereof (e.g., limbus or anterior chamber), resizing images, normalizing illumination or contrast, reducing reflections, and/or enhancing image quality. It should be noted that the surgical image data may include both “still image” and “video sequences.”

The disclosed systems and methods introduce a significant advancement in the field of ophthalmic surgery, specifically addressing challenges encountered during cataract surgery related to the detection and management of RLFs which pose significant risks to the surgical subject. Unlike conventional techniques which rely on experience and visual acuity to identify RLFs which may be hard to detect due to their size, shading, opacity, and/or location, a key feature of the present techniques includes the use of machine learning to analyze images of the surgical procedure to detect and track RLFs. The trained machine learning model offers a high degree of accuracy in detecting RLFs throughout the various conditions (e.g., of the patient anatomy, surgical environment, etc.) and surgical phases of the ophthalmic surgical procedure. The ability to accurately locate, identify, and track RLFs throughout the surgical procedure empowers surgeons to adapt their operative strategies in real-time, potentially reducing surgery duration and improving the efficiency of surgical procedures.

Several improvements allow the computing systems to perform the disclosed techniques. Enhanced networking and processing capability allowing the computer system and machine learning model to provide (near) real-time detection of RLFs while a surgical procedure is on-going, ensuring the identification of RLFs which aid the surgical staff in making critical, immediate decisions to improve the surgical outcome of patients by mitigating complications caused by RLFs. The disclosed systems are configured to provide a combination of advanced image processing, machine learning, and real-time surgical data analysis to identify RLFs that may otherwise remain unnoticed, allowing for removal of RLFs to reduce the likelihood of associated post-surgical complications and enhance the overall safety and efficacy of cataract surgery. The disclosed systems and methods not only facilitate the rapid retrieval and analysis of surgical image data, but also provide the ongoing refinement of machine learning models through retraining as additional training data becomes available. Moreover, the ability to provide (near) real-time surgical image analysis reduces the duration and/or number of surgical procedures and associated surgical computing resources (e.g., processing cycles, memory, network bandwidth, power) to otherwise address undetected RLFs, leading to improved computing performance and better surgical outcomes and patient satisfaction.

In summary, the present techniques represent a significant leap forward in enhancing the safety and efficacy of ophthalmic surgeries via the application of machine learning. Through the aforementioned improvements, the disclosed techniques offer a highly effective approach to mitigating the risks associated with RLFs during ophthalmic surgeries. The disclosed systems and methods present a holistic solution to the aforementioned challenges of convention techniques in effectively detecting and managing residual lens fragments during cataract surgery to provide a significant leap forward in the application of machine learning technologies in the medical field via systems and methods that not only enhance the effectiveness of cataract surgeries but also contributes to safer outcomes and improved post-operative vision for patients. Through its innovative approach, the disclosed techniques allow for technology-assisted surgical procedures which provide advancements in medical technology applications to prioritize patient safety and surgical success.

As used herein, the terms video data and/or image data may refer to data comprising one or more images (e.g., images of a surgical procedure, preprocessed images). Accordingly, at times the terms “video data,” “image data,” and the like may be used interchangeably with the terms “image,” “images,” “video,” and the like.

### Example Computing Environment

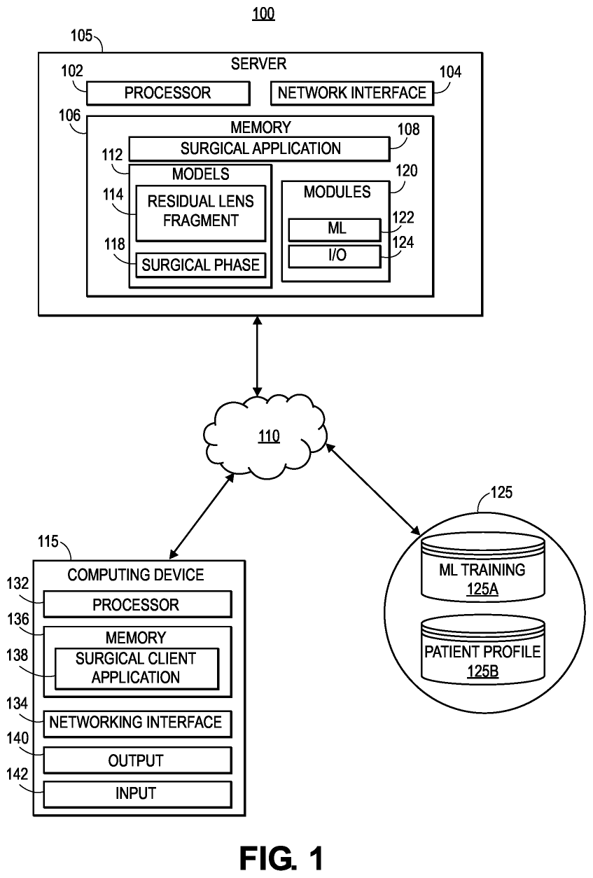

FIG. 1 depicts an example computing environment 100 in which methods and system for detecting a residual lens fragment (RLF) using machine learning (ML) may be implemented, according to embodiments. Although FIG. 1 depicts certain entities, components, equipment, and/or devices, it should be appreciated that additional or alternate entities, components, equipment, and/or devices are envisioned.

The computing environment 100 may include at least one server 105 which may perform the at least some of the functionalities and techniques disclosed, such as obtaining surgical image data, executing machine learning models, detecting RLFs, generating output data associated with detecting RLFs, etc. The server 105, referred to at times more generically as a “computing device” or “device,” may be part of a cloud network or may otherwise communicate with other hardware or software components within one or more cloud computing environments to send, retrieve, or otherwise analyze data or information described herein. In some embodiments, the computing environment 100 may comprise an on-premises computing environment, a multi-cloud computing environment, a public cloud computing environment, a private cloud computing environment, and/or a hybrid cloud computing environment. In one example, an entity may host one or more services (e.g., surgical video analysis) via a public cloud computing environment (e.g., Amazon Web Services (AWS), Google Cloud, IBM Cloud, Microsoft Azure, etc.). The public cloud computing environment may be a traditional off-premises cloud (i.e., not physically hosted at a location owned/controlled by the business). Alternatively, or in addition, aspects of the public cloud may be hosted on-premises at a location owned/controlled by the entity. The public cloud may be partitioned using visualization and multi-tenancy techniques and/or may include one or more of software-as-a-service (SaaS), infrastructure-as-a-service (IaaS) and/or platform-as-a-service (PaaS). In one aspect, the server 105 may include a client-server platform technology such as ASP.NET, Java J2EE, Ruby on Rails, Node.js, a web service or online API, responsive for receiving and responding to electronic requests.

The computing environment 100 may include a network 110 comprising any suitable network or networks, including a local area network (LAN), wide area network (WAN), the Internet, a combination thereof, and/or any other suitable network. Generally, the network 110 enables bidirectional communication between the server 105, the computing device 115, and other components and/or devices of the computing environment 100. In some embodiments, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, or the like. Additionally, or alternatively, the network 110 may comprise one or more routers, wireless switches, or other such wireless connection points communicating to the components of the computing environment 100 via wireless communications based upon any one or more of various wireless standards, including by non-limiting example, IEEE 802.11 a/ac/ax/b/c/g/n (Wi-Fi), Bluetooth, and/or the like.

The server 105 may include at least one processor 102. The processor 102 may include one or more suitable processors (e.g., central processing units (CPUs) and/or graphics processing units (GPUs)). The processor 102 may be communicatively coupled to a memory 106 via a computer bus (not depicted) responsible for transmitting electronic data, data packets, or otherwise electronic signals to and from the processor 102 and memory 106 in order to execute, implement or perform the machine-readable instructions, methods, processes, elements, or limitations, as illustrated, depicted, or described for the various flowcharts, illustrations, diagrams, figures, and/or other disclosure herein. The processor 102 may interface with the memory 106 via the computer bus to execute an operating system (OS) and/or computing instructions contained therein, and/or to access other services/aspects. For example, the processor 102 may interface with the memory 106 via the computer bus to create, read, update, delete, or otherwise access or interact with the data stored in the memory 106, database 125, and/or another source of data.

The computing environment 100 may include a network interface 104. The network interface 104 may allow the server 105 to communicate over the network 110 via any suitable wired and/or wireless connection, e.g., using any suitable network interface controller(s) of the network interface 104. The network interface 104 may include one or more transceivers (e.g., WWAN, WLAN, and/or WPAN transceivers) functioning in accordance with IEEE reference standards, 3GPP reference standards, and/or other reference standards that may be used in receipt and transmission of data via external/network ports of the server 105 connected to computer network 110.

The memory 106 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 106 may store an operating system (OS) (e.g., Microsoft Windows, Linux, UNIX, etc.) capable of facilitating the functionalities, apps, methods, or other software as described herein. The memory 106 may store one or more sets of non-transitory, computer-executable instructions that, when executed, cause the server 105 to perform certain functions.

In general, a computer program or computer based product, application, or code (e.g., the model(s), such as ML models, or other computing instructions described herein) may be stored on a computer usable storage medium, or tangible, non-transitory computer-readable medium (e.g., reference random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 102 (e.g., working in connection with the respective operating system in memory 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C #, Objective C, Java, Scala, ActionScript, JavaScript, HTML, CSS, XML, etc.).

The memory 106 may store a surgical application 108. The surgical application 108 may perform one or more functions associated with detecting RLFs in the eye of a subject, such as obtaining surgical image data of the subject's surgical procedure, providing the surgical image data to one or more of the models 112 (e.g., to detect RLF, identify features of the eye, determine a phase of the surgical procedure), executing one or more of the ML models 112 (e.g., via the ML module 122), generating output data (e.g., user interfaces, surgical video data with informative overlays), etc.

The memory 106, the database 125, and/or other suitable storage may store one or more ML models 112, routines, algorithms, or other elements, referred to herein collectively at times as “models” or “machine leaning models.” Although depicted in FIG. 1 as part of memory 106, one or more of ML models 112 may be considered as one or more computing modules 120. The ML models 112 may be referred to as receiving an input, producing or storing an output, or executing as instructions on the processor 102. Further, the ML models 112 may be stored in the memory 106 and/or the database as executable instructions, which instructions the processor 102 may retrieve from the memory 106 and/or database 125 and execute. Further, the processor 102 should be understood to retrieve from the memory 106 and/or database 125 any data necessary to perform the executed instructions (e.g., data required as an input to the routine or model), and to store in the memory 106 and/or database 125 the intermediate results and/or output of any executed instructions.

In at least one aspect, the ML models 112 may include one or more ML models, as further described below. The ML models 112 may include an RLF model 114. The RLF model 114 may be trained using training data (e.g., stored in the training database 125A) to receive and analyze surgical image data comprising one or more images of an ophthalmic surgical procedure of the subject, and detect at least one RLF within an eye of the subject depicted in the surgical image data. The RLF model 114 may be trained using historical surgical image data (e.g., ophthalmic surgery image data), as further described herein. In at least some embodiments, the RLF model 114 may be trained to identify one or more features of the eye based upon analyzing the surgical image data, for example to differentiate an RLF from another feature of the eye, to indicate the location of the RLF respective to a feature of the eye, etc. The features of the eye may include anatomical features (e.g., the iris, the lens, the pupil, the anterior capsule, the posterior capsule, an iridocorneal angle), features associated with the ophthalmic surgical procedure (an incision in the eye, surgical device proximate the eye), and/or any other suitable feature associated with the eye. The RLF model 114 may identify one or more features of the eye using feature extraction. The RLF model 114 may include one or more of a two-dimensional discrete wavelet transform, higher-order tensor decomposition algorithms (e.g., Tucker algorithm), a higher-order singular value decomposition algorithm, a feature pyramid network, a time series transformer, and/or a convolutional neural network (CNN) (e.g., VGG16 CNN). In at least some embodiments, based upon receiving the surgical image data the RLF model 114 may perform adaptive wavelet tensor feature extraction on one or more images (e.g., to identify features of the subject's eye such as RLFs), may generate one or more images including segmented features of the eye (e.g., of the RLFs, anatomical components of the eye, surgical features of the eye such as incisions), and/or may generate segmentation masks associated with the segmented features, as an output via one or more algorithms (e.g., an empirically optimized algorithm) and/or as part of the process of analyzing the surgical image data to detect the RLFs in the associated images.

The ML models 112 may include a surgical phase model 118. The surgical phase model 118 may be trained using training data to determine one or more phases of a surgical procedure (e.g., the ophthalmic surgical procedure) based upon analyzing the surgical image data. The surgical phase model 118 may be trained using historical surgical image data (e.g., ophthalmic surgery image data), as further described herein. In an example related to a cataract ophthalmic surgery, the surgical phases may include paracentesis, medication injection, viscoelastic insertion, main wound, capsulorrhexis initiation, capsulorrhexis formation, hydrodissection, phacoemulsification, irrigation and aspiration, cortical removal, lens insertion, viscoelastic removal, and/or wound closure. In at least some embodiments, particular phases of surgery may be associated with RLF detection criteria. For example, the server 105 and/or other suitable computing device may determine whether an RLF meets one or more detection criteria, which may include detecting the RLF during a particular phase of the ophthalmic surgical such as phacoemulsification, cortical removal, viscoelastic insertion, lens insertion, viscoelastic removal, or wound closure.

The memory 106 may store one or more computing modules 120, implemented as respective sets of computer-executable instructions (e.g., one or more source code libraries) as described herein. The computing modules 120 may include an ML module 122. In some embodiments, ML models (e.g., the ML model 112) may be applied by the ML module 122, which may include, but are not limited to linear or logistic regression algorithms, instance-based algorithms, regularization algorithms, decision trees, Bayesian networks, cluster analysis, association rule learning, artificial neural networks, deep learning, combined learning, reinforcement learning, dimensionality reduction, and support vector machines. In various embodiments, the implemented ML methods and algorithms are directed toward at least one of a plurality of categorizations of ML, such as supervised learning, unsupervised learning, and reinforcement learning. In one aspect, the ML based algorithms may be included as a library or package executed on server(s) 105. For example, libraries may include the TensorFlow based library, the Pytorch library, and/or the scikit learn Python library.

In one embodiment, the ML module 122 employs supervised learning, which involves identifying patterns in existing data to make predictions about subsequently received data. Specifically, the ML module 122 is “trained” using training data, which includes exemplary inputs and associated exemplary outputs. Based upon the training data, the ML module 122 may generate a predictive function which maps outputs to inputs and may utilize the predictive function to generate ML outputs based upon data inputs. The exemplary inputs and exemplary outputs of the training data may include any of the data inputs or ML outputs described herein. In the exemplary embodiments, a processing element may be trained by providing it with a large sample of data with known characteristics or features.

In another embodiment, the ML module 122 may employ unsupervised learning, which involves finding meaningful relationships in unorganized data. Unlike supervised learning, unsupervised learning does not involve user-initiated training based upon exemplary inputs with associated outputs. Rather, in unsupervised learning, the ML module 122 may organize unlabeled data according to a relationship determined by at least one ML method/algorithm employed by the ML module 122. Unorganized data may include any combination of data inputs and/or ML model outputs.

In yet another embodiment, the ML module 122 may employ reinforcement learning, which involves optimizing outputs based upon feedback from a reward signal. Specifically, the ML module 122 may receive a user-defined reward signal definition, receive a data input, utilize a decision making model to generate the ML output based upon the data input, receive a reward signal based upon the reward signal definition and the ML output, and alter the decision making model so as to receive a stronger reward signal for subsequently generated ML outputs. Other types of ML may also be employed, including deep or combined learning techniques.

The ML module 122 may receive labeled data at an input layer of a model having a networked layer architecture (e.g., an artificial neural network, a convolutional neural network, etc.) for training one or more ML models. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models.

The ML module 122 may comprise a set of computer-executable instructions to implement functionality such as loading, configurating, initializing, operating, and/or storing (e.g., in the memory 106, the database 125) the ML model 112. Once trained, one or more of the trained ML model 112 may be operated in inference mode, whereupon when provided with de novo input that the model has not previously been provided, the model may output one or more predictions, classifications, etc., as described herein.

In operation, the ML module 122 may access the memory 106, the training database 125A, and/or any other data source for training data suitable to generate one or more ML models, such as the ML model 112. The training data may be sample data with assigned relevant and comprehensive labels (classes or tags) used to fit the parameters (weights) of the ML model with the goal of training it by example. In one aspect, once an appropriate ML model is trained and validated to provide accurate predictions and/or responses, the trained ML model may be loaded into the ML module 122 at runtime to process input data and generate output data.

While various embodiments, examples, and/or aspects disclosed herein may include training and generating the ML model 112 for the server 105 to load at runtime, one or more appropriately trained ML models may already exist (e.g., stored in the memory 106, the database 125) such that the server 105 may load the existing trained ML model 112 at runtime. The server 105 may retrain, fine-tune, update and/or otherwise alter an existing ML model 112 before and/or after loading the ML model 112 at runtime. Although the ML model 112 may be described as being trained and operated (e.g., via ML module 122) on the server 105, in at least one embodiment the ML model 112 may be trained on a first server 105 or other first computing device, and operated on another second server or second computing device.

In one aspect, the computing modules 120 may include an input/output (I/O) module 124, comprising a set of computer executable instructions implementing communication functions. The I/O module 124 may include a communication component configured to communicate (e.g., send and receive) data via one or more external/network port(s) to one or more networks or local terminals, such as the computer network 110 described herein. In one aspect, the server 105 may include a client-server platform technology such as ASP. NET, Java J2EE, Ruby on Rails, Node. js, a web service or online API, responsive for receiving and responding to electronic requests.

The I/O module 124 may further include or implement a user interface configured to present information to an administrator, operator or other user, and/or receive inputs from the user, such as via a touchscreen display. The I/O module 124 may facilitate I/O components (e.g., ports, capacitive or resistive touch sensitive input panels, keys, buttons, lights, LEDs), which may be directly accessible via, or attached to, the server 105 and/or may be indirectly accessible via, or attached to, another device. According to one aspect, a user may access the server 105 via a user interface to review information, make changes, input training data, initiate ML model training via the ML module 122, and/or perform other functions (e.g., operation of one or more trained models via the ML module 122).

The network 110 may include one or more networks, including a local area network (LAN), wide area network (WAN), the Internet, a combination thereof, and/or any other suitable network. Generally, the network 110 enables bidirectional communication between the server 105, the computing device 115, and other components and/or devices of the computing environment 100. In some embodiments, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, or the like. Additionally, or alternatively, the network 110 may comprise one or more routers, wireless switches, or other such wireless connection points communicating to the components of the computing environment 100 via wireless communications based upon any one or more of various wireless standards, including by non-limiting example, IEEE 802.11 a/ac/ax/b/c/g/n (Wi-Fi), Bluetooth, and/or the like.

The computing environment 100 may include at least one computing device 115, which may be referred to at times as a “user device.” The computing device 115 may comprise one or more computers and/or multiple, redundant, or replicated client computers accessed by one or more users. The computing device 115 may include a computer (e.g., desktop computer, laptop computer, terminal, server), a mobile device, a wearable, augmented reality glasses/headsets, virtual reality glasses/headsets, mixed or extended reality glasses/headsets, a surgical computing device (e.g., surgical computer, surgical display, surgical headset, surgical tool), and/or other suitable computing device. The computing device 115 may be associated with, for example, one or more surgical staff performing the ophthalmic surgical procedure on the subject, such as a surgical tool or camera that captures surgical image data of a surgical procedure, a surgical display that displays the surgical image data (e.g., in real-time during the surgical procedure) with additional information, a surgical headset used by a surgeon to assist with the surgical procedure (e.g., providing guidance during the surgical procedure), etc. The computing device 115 may include a processor 132 (e.g., the processor 102), a network interface 134 (e.g., the network interface 104), and a memory 136 (e.g., the memory 106. The computing device 115 may include at least one output component 140 associated with generating an output at the computing device 115. The output component 140 may include a visual component (e.g., LCD, LED, OLED, and/or head-mounted display, a light, a touchscreen, etc.), an audio component (e.g., speaker), a feedback component (e.g., haptic motor), and/or other suitable output component. The computing device 115 may include at least one input component 142 associated with receiving an input at the computing device 115, such as an a mouse, keyboard, touchscreen, a sensor, a camera (e.g., charge-coupled device, complementary metal-oxide semiconductor), and/or other suitable input component. The computing device 115 may access services, devices, and/or components of the computing environment 100 via the network 110 (e.g., transmitting data over the network 110 via the network interface 134). In some embodiments, the computing device 115 transmits to, and/or receives data from, the server 105, the database 125, and/or other components of the computing environment 100. For example, the computing device 115 may capture surgical image data via the input component 142 (e.g., camera), store the surgical image data in the patient database 125B, transmit the surgical image data to the server 105 for analysis via one or more of the models 112, etc.

In at least some embodiments, the memory 136 of the computing device 115 may store a surgical client application 138. The surgical client application 138 may be configured to provide the same and/or similar functionality as the surgical application 108. The surgical client application 138 may be in communication (e.g., via the network 110) with the surgical application 108 to provide at least some of the functionality of the surgical application 108 to the user of the computing device 115. In at least some embodiments, the surgical client application 138 may be installed on computing devices 115 used during a surgical procedure such as an ophthalmic surgical procedure, for example to provide surgical guidance, warnings, provide visual information (e.g., surgical images overlaid with information), etc.

The computing environment 100 may include at least one electronic database 125. The server 105, the computing device 115, and/or other devices and/or components of the computing environment 100 may be communicatively coupled (e.g., via network 110) to the database 125. The database 125 may include a relational database, such as Oracle, DB2, MySQL, a NoSQL database such as MongoDB, and/or another other suitable database. The database 125 may store data and/or datasets accessible to components and devices of the computing environment 100 (e.g., the server 105, the computing device 115) via the network 110. As used herein, the term “dataset” may describe sets of data composed of separate elements (e.g., files), however, the terms “data” and “dataset(s)” may be used interchangeably herein.

The database 125 may include an ML training database 125A storing training data/datasets associated with training one or more of the ML models 112. The database 125 may include a patient database 125B storing data associated with surgical patients, for example for storing surgical image data of a surgical procedure on a surgical patient in their respective patient profile in the patient database 125B.

In operation, the server 105 may obtain surgical image data comprising a plurality of images capturing an ophthalmic surgical procedure of a subject. A computing device computing device 115, such as a surgical computer having a camera, may record the surgical image data in substantially real-time which the computing device 115 provides to the server 105 via the network 110. The surgical application 108 may provide the surgical image data to the RLF model 114 and in response the RLF model 114 may analyze the surgical image data detect at least one RLF within an eye of the subject in the associated images, which may include detecting characteristics of the RLF (e.g., size, shape, location), semantically segmenting the RLF in the associated image of the surgical image data, etc. The surgical application 108 (e.g., via the RLF model 114) may determine whether the detected RLF meets at least one detection criteria.

In at least some embodiments, the detection criteria includes the location of the at least one RLF respective to the one or more features of the eye (e.g., an incision, an iris, an anterior capsule, a posterior capsule, or an iridocorneal angle). The RLF model 114 may identify one or more features of the eye based upon analyzing the surgical image data, and the surgical application 108 may determine a location of the at least one RLF respective to the one or more features of the eye to determine whether the detected RLF meets at least one detection criteria.

In at least some embodiments, the detection criteria includes detecting the at least one RLF during the particular phase of the ophthalmic surgical procedure (e.g., phacoemulsification, cortical removal, viscoelastic insertion, lens insertion, viscoelastic removal, or wound closure). The surgical phase model 118 may determine one or more phases of the ophthalmic surgical procedure based upon analyzing the surgical image data, and the surgical application 108 may determine the RLF is detected during a particular phase of the one or more phases of the ophthalmic surgical procedure, to determine whether the detected RLF meets at least one detection criteria. For example, the surgical application 108 may detect when RLFs remain in the eye of the subject after the completion of the phacoemulsification and cortical removal phases of surgery.

Based upon determining the at least one RLF meets the at least one detection criteria, the surgical application 108 generate output data associated with detecting the at least one RLF in the eye of the subject, and transmit the output data to the computing device 115. However, in at least some embodiments, the surgical application 108 may generate the output data based upon the RLF model 114 detecting one or more RLFs, without requiring one or more of the detected RLFs to meet detection criteria. The output data may include a suggestion to polish the anterior capsule or posterior capsule, an indication of the size and/or location of the at least one RLF. For example, when the surgical application 108 detects RLFs after the completion of the phacoemulsification and cortical removal phases of surgery, the output data when received by the computing device 115 may cause display of an alert to the surgeon as to the presence of the RLFs including a visualization indicating the location of the RLFs, to aid in their removal and reducing potential morbidity or other complications for the surgical subject.

In at least some embodiments, generating the output data may include generating composite surgical image data by generating an overlay on one or more images of the surgical image data, wherein the overlay is associated with visually identifying the at least one RLF that meets at least one detection criteria in the one or more images. The output data may include the composite surgical image data.

Although the computing environment 100 is shown to include one each of the server 105, the network 110, and the computing device 115, it should be understood that different numbers of servers 105, networks 110 and/or computing devices 115 may be utilized. In one example, the computing environment 100 may include dozens of servers 105 all of which may be interconnected via the network 110 to hundreds of computing devices 115.

The computing environment 100 may include additional, fewer, and/or alternate components, and may be configured to perform additional, fewer, or alternate actions, including components/actions described herein. For example, although the server 105 is shown in FIG. 1 as including one instance of various components such as the processor 102, the memory 106 and the database 125, various aspects include the computing environment 100 and/or the server 105 implementing any suitable number of any of the components shown in FIG. 1 and/or omitting any suitable ones of the components shown in FIG. 1. For instance, information described as being stored in the database 125 may be stored in the memory 106, and therefore the database 125 may be omitted. Moreover, various aspects include the computing environment 100 having any suitable number of additional component(s) not shown in FIG. 1, such as but not limited to the example components described above. Furthermore, it should be appreciated that additional and/or alternative connections between components shown in FIG. 1 may be implemented. As just one example, server 105 may have a direct connection to database 125 rather than appearing to be connected via the network 110 as illustrated in FIG. 1.

### Example ML Model Training

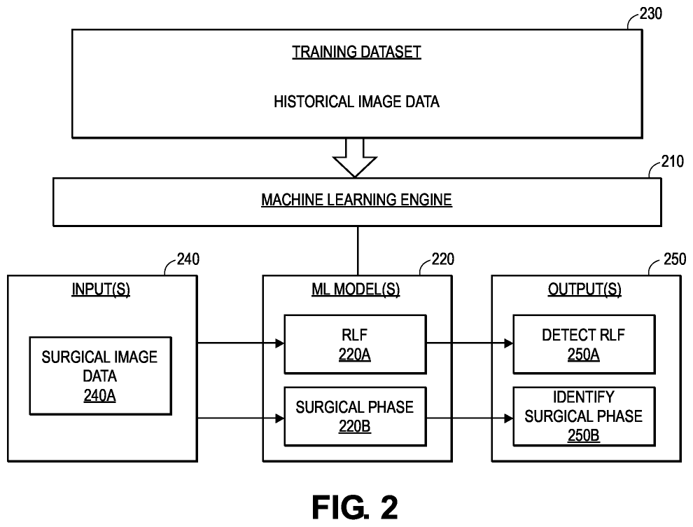

FIG. 2 depicts a combined block and logic diagram for training a machine learning model, according to embodiments. More specifically, an ML engine 210 (e.g., the ML module 122) trains one or more ML model 220 (e.g., the ML model 112) using training data 230 (e.g., stored in the training database 125A). The trained ML model 220 may be applied to, and/or receive, at least one input 240 and generate at least one output 250.

The ML engine 210 may include one or more hardware and/or software components (e.g., the ML module 122) to obtain, create, operate, train, retrain, fine-tune, and/or store one or more ML models 220. It should be understood that at times the term “train” may be used interchangeably with the terms “retrain” and/or “fine-tune.” A server (e.g., the server 105), may obtain and/or have available (e.g., stored in the database 125) one or more types of training data 230 for model creation, training, retraining and/or fine-tuning (generally referred to herein as “training”). In at least one aspect, at least some of the training data 230 may be labeled to aid in training the ML model 220. The ML engine 210 may process and/or analyze the training data 230 to learn associations and/or relationships in the training data 230, and configure the ML model 220 to process the training data 230 such that when the ML model 220 receives one or more inputs 240, the ML model 220 generates appropriate output(s) 250. The ML model 220 may be trained via regression, k-nearest neighbor, support vector regression, and/or random forest algorithms, although any type of applicable ML algorithm and/or training may be used, including training using one or more of supervised learning, unsupervised learning, semi-supervised learning, and/or reinforcement learning.

In at least one aspect, the ML model 220 may be considered as successfully trained when able to achieve one or more metrics (e.g., a score indicating accuracy of the output 250) associated with its performance when processing the training data 230. Once trained, the ML engine 210 may load the ML model 220 at runtime to perform operations on one or more data inputs 240 to produce the desired data output 250.

In at least some embodiments, the ML model 220 includes an RLF model 220A (e.g., the RLF model 114) that may be trained via the ML engine 210 using the training data 230 to receive as an input 240 surgical image data 240A and in response detect or otherwise identify at least one RLF 250A within an eye of the subject (e.g., during one or more phases of an ophthalmic surgical procedure) depicted in the surgical image data 240A as the output 250. The training data 230 for the RLF model 220A may include historical image data comprising a plurality of images (e.g., capturing an ophthalmic surgical procedure) of a plurality of historical subjects, and/or any other suitable training data 230. The historical images may not include RLFs, may include RLFs that are not identified (e.g., via tags, labels, metadata, etc.), and may include RLFs of the eyes of historical subjects that are identified, and/or other suitable images for training the RLF model 220A. The RLF model 220A may be trained to make associations in the training data 230 between objects in the historical image data that may be RLFs, such that when receiving the surgical image data 240A as the input, the RLF model 220A is able to detect RLFs 250A in the surgical image data 240A as the output 250. For example, the RLF model 220A may detect an object in the surgical image data 240A as being an RLF based upon the size, shape, location, opacity, thickness, and/or other characteristics of the object, and may track the RLFs throughout the surgical images of the surgical image data 240A.

In at least some embodiments, the RLF model 220A may be trained to detect or otherwise identify at least one feature of the eye (e.g., anatomical features, surgical features, etc.) when detecting RLFs in the surgical image data 240A. In some such embodiments, the historical image data used for training the RLF model 220A may indicate (e.g., via tags, labels, metadata, etc.) one or more features of the eye of historical subjects, such that the RLF model 220A may be trained to make associations in the training data 230 with objects in the historical images and their associated features, such that when receiving the surgical image data 240A as the input the RLF model 220A is further able to detect one or more features of the eye in the images of the surgical image data 240A. The RLF model 220A may detect or otherwise identify an object as a feature of the eye in the surgical image data based upon the size, shape, location (e.g., in relation to other features such as a surgical tool or anatomical feature), color, and/or other characteristics of the object, and/or be trained in any suitable manner to detect at least one feature of the eye.

In some embodiments, prior to providing the surgical image data to the machine learning model, the system performs preprocessing operations on the images. Such preprocessing may include identifying and extracting a region of interest (ROI) corresponding to the eye, such as the limbus, anterior chamber, or lens region. The ROI may be resized (e.g., to the predefined resolution), normalized, or enhanced to improve visibility of RLFs.

Additional preprocessing may include contrast normalization, illumination correction, noise reduction, and removal of specular reflections associated with surgical lighting or instruments. These preprocessing steps may improve the robustness and accuracy of the machine learning model in detecting small or low-contrast residual lens fragments.

In at least some embodiments, the ML model 220 includes a surgical phase model 220B (e.g., the surgical phase model 118) that may be trained via the ML engine 210 using the training data 230 to receive as an input 240 surgical image data 240A and in response surgical determine the one or more phases of the surgical procedure 250B depicted in the surgical image data 240A as the output 250. The training data 230 for the surgical phase model 220B may include historical image data comprising a plurality of images (e.g., capturing an ophthalmic surgical procedure of a plurality of historical subjects), and/or any other suitable training data 230. At least some of the historical images may indicate or otherwise identify (e.g., via tags, labels, metadata, etc.) the phase of the surgical procedure. The surgical phase model 220B may be trained to make associations in the training data 230 between objects in the historical images that may be indicative of a phase of a surgical procedure, such as the condition and/or features of the eye and/to the presence or absence of particular surgical tools, identify the surgical phase based upon identifying other (e.g., subsequent) phases of the surgical procedure, etc. For example, the surgical phase model 220B may detect an irrigation surgical tool in the images of the surgical image data 240A, and/or detecting characteristics of the eye indicative of irrigation (e.g., fluids in the eye), and determine the phase of a cataract surgery surgical procedure 250B is the irrigation and aspiration phase.

The server and/or the ML engine 210 may update the training data 230 at one or more times. The ML model 220 may be retrained based upon the updated training data 230, the retrained/updated ML model 220 may be stored in memory (e.g., the memory 106) and/or subsequently executed based upon the retraining. Model retraining process may cause the output 250 of the ML model 220 to improve over time. For example, the training data 230 may be updated to include new images of ophthalmic surgical procedures associated with a medication injection phase of the surgical procedure such the after retraining the surgical phase model 220B using the updated training data, the ability of the surgical phase model 220B to detect the medication injection phase of surgical procedure 250B improves. In another example, a surgeon may provide feedback associated with detection of RLFs and/or identification of eye features provided by the RLF model 220A, such as feedback regarding whether particular RLFs and/or eye features were detected and identified. The updated training data 230 may incorporate the surgeon's feedback to retrain and improve the performance of the RLF model 220A respective to the detection of RLFs and/or identification of eye features.

In at least some embodiments, one or more of the models 220 may receive inputs 240 and/or generate outputs 250 other than those depicted in FIG. 2. Accordingly, the inputs 240 and outputs 250 should be consider as at least one embodiment of the disclosed techniques. For example, the RLF model 220A and the surgical phase model 220B may all receive the same surgical image data 240A as the input 240. The RLF model 220A may identify features of the eye in the surgical image data 240A when detecting RLFs 250A. The identified features of the eye may be provided as an additional input to the surgical phase model 220B to improve the accuracy of the determination of the phase of the surgical procedure 250B. For example, the RLF model 220A may identify the lens cortex in a portion of the surgical image data 240A which, when provided as an additional input to the surgical phase model 220B, improves the ability of the surgical phase model 220B to identify the surgical phase surgical procedure 250B associated with the portion of the surgical image data as the cortical removal phase. Accordingly, the training data 230 of the surgical phase model 220B may further include historical surgical image data wherein at least some images identify one or more features of the eye.

It should be understood that functionality that is described as being associated with one or more particular models may be attributed to one or more other models. For example, a third model, which is not the RLF model 220A or the surgical phase model 220B, may be trained to identify features of the eye based upon receiving the surgical image data 240A, such that the RLF model 220A is not trained to identify features of the eye other than RLFs as the output 250.

### Example System for Detection of a Residual Lens Fragment Using Machine Learning

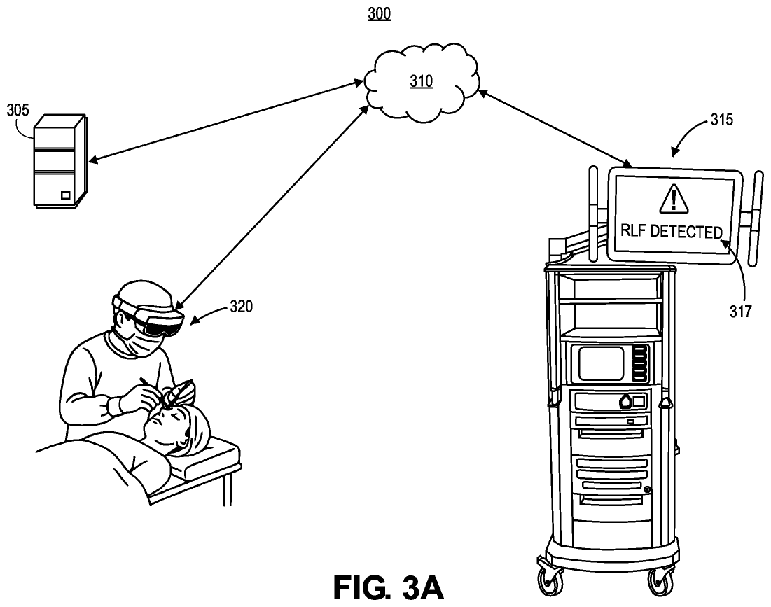

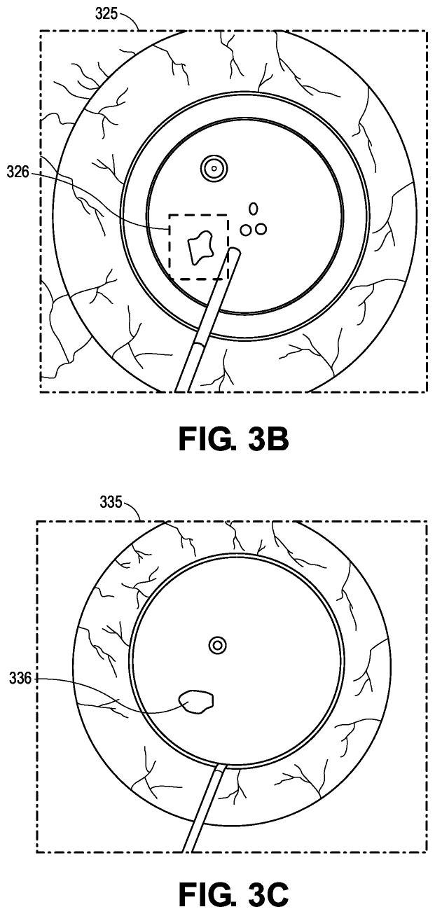

FIG. 3A depicts an example system 300 for detecting a residual lens fragment using machine learning, according to embodiments. In general, the system 300 include devices, models, and/or components of the computing environment 100. The system 300 includes a server 305 (e.g., the server 105), a network 310 (e.g., the network 110), a surgical computer 315 (e.g., the computing device 115) having a display (e.g., the at least one output component 140, a 3D display for viewing with 3D glasses), and a surgical headset 320 (e.g., the computing device 115) worn by a surgeon performing an ophthalmic surgical procedure, such as cataract surgery, on a subject. The surgical headset 320 may include an augmented reality (AR) headset, a 3D display within the oculars of the surgical microscope, and/or any other suitable surgical display. One or more cameras (e.g., the input component 142) of the surgical headset 320 may capture real-time surgical video (e.g., surgical image data) of the ophthalmic surgical procedure. The surgical headset 320 may transmit the surgical video via the network 310 to the surgical computer 315 for display, for example to provide information (e.g., real-time video of the surgery) associated with the surgical procedure. The surgical headset 320 and/or the surgical computer 315 may transmit the surgical video via the network 310 to the server 305 for analysis by a surgical analysis application (e.g., the surgical application 108), for example to detect RLFs in the eye of the subject during the surgical procedure and/or generate surgical guidance based there upon.

The server 305 may store and/or have access to one or more models (e.g., the ML models 112, 220) to analyze the surgical video via the surgical analysis application. The models may include an RLF model that, upon receiving the surgical video as an input, detects one or more residual lens fragments (RLFs) in one or more images depicting the eye of the subject during the ophthalmic surgical procedure. Once the RLF model detects one or more RLFs in the eye of the subject depicted in the surgical video, the RLF model may track the RLFs throughout the surgical procedure via the surgical video. It should be understood that one or more of the surgical computer 315 or surgical headset 320 may store and/or have access to a surgical analysis application (e.g., the surgical client application 138) and/or one or more model, such that the surgical computer 315 and/or surgical headset 320 may process and/or analyze the surgical video locally on the respective device rather than transmit the surgical video to the server 305 for analysis.

FIG. 3B depicts an example first image 325 of a surgical video capturing an ophthalmic surgical procedure of a subject, according to embodiments. The surgical headset 320 (e.g., via a camera, input component 142) may capture the surgical video including the first image 325. The server 305 via the surgical analysis application may provide the surgical video including the first image 325 to an RLF model stored on the server 305. Based upon receiving the surgical video, the RLF model may detect an RLF 326 in the first image 325.

The surgical analysis application may determine (e.g., via the RLF model and/or other functionality of the surgical analysis application) whether a detected RLF meets one or more detection criteria. The detection criteria may be associated with characteristics of the RLF, such as the RLF shape, the RLF size, the lens layer which the RLF is from, and/or location within of the RLF the eye (e.g., within the anterior chamber, posterior chamber, within the lens capsule, posterior to the lens capsule in the vitreous cavity). For example, the RLF model and/or otherwise surgical application may apply clarity analysis algorithms with capsulotomy morphology analysis algorithms to, among other things, detect an extremely small RLF created during the surgical procedure from a layer of lens which is easily absorbed by the eye if small enough. Thus, the RLF may not meet detection criteria due to the size and lens layer associated with the RLF, and the surgical application may not provide any feedback related to the small RLF.

In at least some embodiments, to determine whether the RLF meets detection criteria the surgical application may identify (e.g., via the RLF model) one or more features of the eye based upon analyzing the surgical image data. The features of the eye may include an incision in the eye, the iris, the anterior capsule, the posterior capsule, an iridocorneal angle, and/or any other suitable feature of the eye. The detection criteria may be associated with the location of the RLF, and the surgical application may determine a location the RLF respective to the identified features of the eye. For example, the RLF model may identify the lens capsule and identify the location of the RLF as within an incision created in the eye.

In at least some embodiments, to determine whether the RLF meets detection criteria the surgical application may determine (e.g., via the surgical phase model 118, 220B) one or more phases of the ophthalmic surgical procedure based upon analyzing the surgical image data. The detection criteria may be associated with detecting the RLF during a particular phase of the surgical procedure, such as phacoemulsification, cortical removal, viscoelastic insertion, lens insertion, viscoelastic removal, or wound closure. For example, the criteria may be associated with detecting the RLF during a phase of surgery where all RLFs should have already been removed from the eye of the subject.

Based upon determining the RLF meets the at least one detection criteria, the surgical analysis application may generate output data associated with detecting the RLF that meets the detection criteria. The output data may include a graphical user interface, a notification (e.g., audio, visual), surgical images with information overlaid, etc. The output data may indicate the size, shape, location, number, position, depth, and/or other characteristics of one or more detected RLFs, surgical guidance (e.g., suggestions to remove the RLFs), risks to the patient associated with the RLFs, and/or any other suitable output providing information associated with the RLFs. The server 305 may transmit the output data via the network 310 to the surgical computer 315 and/or the surgical headset 320. The surgical computer 315 and/or surgical headset 320 (e.g., via a surgical client application 138) may output the output data, for example in real-time during the surgical procedure to provide a warning 317 and/or guidance to the surgeon.

FIG. 3C depicts an example second image 335 of a surgical video including a semantically segmented RLF 336, according to embodiments. The surgical application may generate the second image 335 by overlaying the segmentation mask associated with the segmented RLF onto an image (e.g., the first image 325) of the surgical video. The segmentation mask may assist a viewer of the image in visually identifying the location of the RLF. The server 305 via the surgical application may transmit the second image 335 to the surgical computer 315 to be output (e.g. via the surgical client application) on the display of the surgical computer 315.

In at least some embodiments, the surgical headset 320 may execute the surgical client application to generate and/or otherwise obtain (e.g., from the server 305) information associated with the detected RLF, the information displayed on an AR display of the surgical headset 320. For example, information indicating the size, shape and location of one or more RLFs may be overlaid in real-time on the surgical video to provide real-time surgical guidance to the surgeon wearing the surgical headset 320 to aid in removal of the RLFs from the eye of the patient.

### Example Computer-Implemented Method for Detecting a Residual Lens Fragment Using Machine Learning

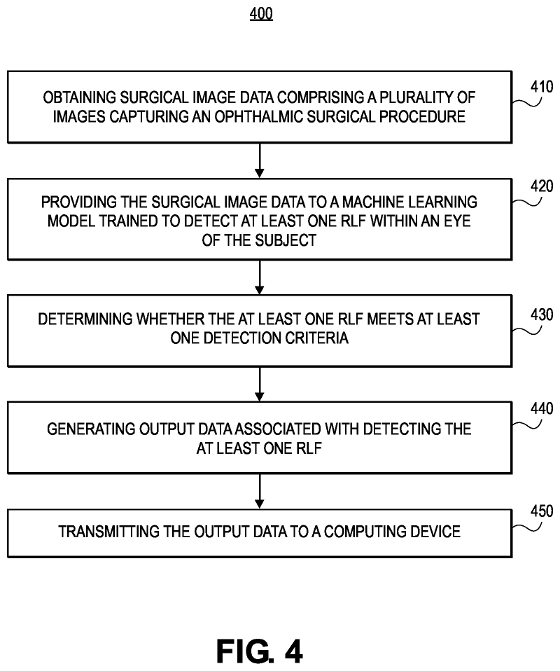

FIG. 4 is an example block flow diagram depicting a computer-implemented method 400 for detecting a residual lens fragment (RLF) using machine learning, according to embodiments. In general, the computer-implemented method 400 may be performed via the computing environment 100 and/or the system 300. One or more steps of the computer-implemented method 400 may be implemented as a set of instructions stored on a computer-readable memory (e.g., the memory 106, the memory 136) and executable by one or more processors (e.g., the processor 102, the processor 132).

The computer-implemented method 400 may include obtaining surgical image data (e.g., video) comprising a plurality of images capturing an ophthalmic surgical procedure (e.g., a cataract surgery) of a subject (block 410).

The computer-implemented method 400 may include providing the surgical image data to a machine learning model (e.g., the RLF model 114, 220A) trained to detect at least one RLF within an eye of the subject during the ophthalmic surgical procedure based upon analyzing the surgical image data (block 420).

The computer-implemented method 400 may include determining whether the at least one RLF meets at least one detection criteria (block 430) (e.g., a size of the RLF, layer of lens of the RLF, a location of the RLF, surgical phase of detection). In at least some embodiments, computer-implemented method 400 may include identifying (e.g., via the RLF model) one or more features of the eye (e.g., an incision, an iris, an anterior capsule, a posterior capsule, and/or an iridocorneal angle), and determining a location of the at least one RLF respective to the one or more features of the eye, wherein the at least one detection criteria includes the location of the at least one RLF respective to the one or more features of the eye. The output data may include or otherwise indicate one or more of a suggestion to polish the anterior capsule, polish the posterior capsule, or an indication (e.g., a semantically segmented RLF) of the location of the at least one RLF.

In at least some embodiments, determining the at least one RLF meets at least one detection criteria may include determining one or more phases of the ophthalmic surgical procedure based upon analyzing the surgical image data, and determining the at least one RLF is detected during a particular phase of the one or more phases of the ophthalmic surgical procedure, wherein the at least one detection criteria includes detecting the at least one RLF during the particular phase of the ophthalmic surgical procedure. In some such embodiments, a second model (e.g., the surgical phase model 118, 220B) may determine one or more phases of the ophthalmic surgical procedure based upon analyzing the surgical image data and/or the particular phase of the ophthalmic surgical procedure may include one or more of phacoemulsification, irrigation and aspiration, wound closure, or cortical removal.

The computer-implemented method 400 may include, based upon determining the at least one RLF meets the at least one detection criteria (block 430), generating output data associated with detecting the at least one RLF in the eye of the subject (block 440).

The computer-implemented method 400 may include transmitting the output data to a computing device (e.g., the computing device 115, the surgical computer 315, the surgical headset 320) (block 450). Generating the output data (block 440) may include generating composite surgical image data by generating an overlay on one or more images of the plurality of images of the surgical image data, wherein the overlay is associated with visually identifying the at least one RLF that meets at least one detection criteria in the one or more images.

In at least some embodiments, the computer-implemented method 400 may include detecting the at least one RLF within the eye of the subject by the machine learning model may include detecting a size of the at least one RLF wherein the output data may indicate the size of the at least one RLF, and/or detecting a layer of a lens of the eye of the subject associated with the at least one RLF, wherein the output data may indicate the layer of the lens associated with the at least one RLF.

It should be understood that not all blocks of the example flow diagram of FIG. 4 are required to be performed.

### Additional Considerations

With the foregoing, users whose data is being collected and/or utilized may first opt-in. After a user provides affirmative consent, data may be collected from the user's device (e.g., a mobile computing device). In other embodiments, deployment and use of ML models at a client or computing device may have the benefit of removing any concerns of privacy or anonymity, by removing the need to send any personal or private data to a remote server.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S.C. § 114(f) unless traditional means-plus-function language is expressly recited, such as “means for” or “step for” language being explicitly recited in the claim(s). The systems and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment”, “in one aspect” and/or the like in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory product to which it is communicatively coupled. A further hardware module may then, at a later time, access the memory product to retrieve and process the stored output. Hardware modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a building environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Upon reading this disclosure, those of skill in the art will appreciate still additional alternative structural and functional designs for the method and systems described herein through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

Thus, many modifications and variations may be made in the techniques, methods, and structures described and illustrated herein without departing from the spirit and scope of the present claims. Accordingly, it should be understood that the methods and apparatus described herein are illustrative only and are not limiting upon the scope of the claims.

What is claimed:

1. A computer-implemented method for detecting a residual lens fragment (RLF) using machine learning, the computer-implemented method comprising:
obtaining, by one or more processors, surgical image data comprising a plurality of images capturing an ophthalmic surgical procedure of a subject;
providing, by the one or more processors, the surgical image data to a machine learning model trained to detect at least one RLF within an eye of the subject during the ophthalmic surgical procedure based upon analyzing the surgical image data;
determining, by the one or more processors, whether the at least one RLF meets at least one detection criteria;
based upon determining the at least one RLF meets the at least one detection criteria, generating, by the one or more processors, output data associated with detecting the at least one RLF in the eye of the subject; and
transmitting, by the one or more processors, the output data to a computing device.

2. The computer-implemented method of claim 1, wherein determining the at least one RLF meets at least one detection criteria comprises:
identifying, by the one or more processors, one or more features of the eye based upon analyzing the surgical image data; and
determining, by the one or more processors, a location of the at least one RLF respective to the one or more features of the eye,
wherein the at least one detection criteria includes the location of the at least one RLF respective to the one or more features of the eye.

3. The computer-implemented method of claim 2, wherein the one or more features of the eye include one or more of: an incision, an iris, an anterior capsule, a posterior capsule, or an iridocorneal angle.

4. The computer-implemented method of claim 3, wherein the output data includes a suggestion to polish the anterior capsule or posterior capsule.

5. The computer-implemented method of claim 2, wherein the output data includes an indication of the location of the at least one RLF.

6. The computer-implemented method of claim 5, wherein the indication of the location of the at least one RLF includes a semantically segmented RLF.

7. The computer-implemented method of claim 1, wherein determining the at least one RLF meets at least one detection criteria comprises:
determining, by the one or more processors, one or more phases of the ophthalmic surgical procedure based upon analyzing the surgical image data; and
determining, by the one or more processors, the at least one RLF is detected during a particular phase of the one or more phases of the ophthalmic surgical procedure,
wherein the at least one detection criteria includes detecting the at least one RLF during the particular phase of the ophthalmic surgical procedure.

8. The computer-implemented method of claim 7, wherein a second model determines the one or more phases of the ophthalmic surgical procedure based upon analyzing the surgical image data.

9. The computer-implemented method of claim 7, wherein the particular phase of the ophthalmic surgical procedure includes one or more of: phacoemulsification, cortical removal, viscoelastic insertion, lens insertion, viscoelastic removal, or wound closure.

10. The computer-implemented method of claim 1, wherein detecting the at least one RLF within the eye of the subject by the machine learning model includes:
detecting a size of the at least one RLF,
wherein the output data indicates the size of the at least one RLF.

11. The computer-implemented method of claim 1, wherein detecting the at least one RLF within the eye of the subject by the machine learning model includes:
detecting a layer of a lens of the eye of the subject associated with the at least one RLF,
wherein the output data indicates the layer of the lens associated with the at least one RLF.

12. The computer-implemented method of claim 1, wherein generating the output data comprises:
generating, by the one or more processors, composite surgical image data by generating an overlay on one or more images of the plurality of images of the surgical image data,
wherein the overlay is associated with visually identifying the at least one RLF that meets at least one detection criteria in the one or more images.

13. A system for detecting a residual lens fragment (RLF) using machine learning, the system comprising:
one or more processors; and
one or more non-transitory memories storing processor-executable instructions that, when executed by the one or more processors, cause the system to:
obtain surgical image data comprising a plurality of images capturing an ophthalmic surgical procedure of a subject;
provide the surgical image data to a machine learning model trained to detect at least one RLF within an eye of the subject during the ophthalmic surgical procedure based upon analyzing the surgical image data;
determine whether the at least one RLF meets at least one detection criteria;
based upon determining the at least one RLF meets the at least one detection criteria, generate output data associated with detecting the at least one RLF that meets at least one detection criteria; and
transmit the output data to a computing device.

14. The system of claim 13, wherein to determine the at least one RLF meets at least one detection criteria comprises instructions that, when executed by the one or more processors, cause the system to:
identify one or more features of the eye based upon analyzing the surgical image data; and
determine a location of the at least one RLF respective to the one or more features of the eye,
wherein the at least one detection criteria includes the location of the at least one RLF respective to the one or more features of the eye.

15. The system of claim 14, wherein the one or more features of the eye include one or more of: an incision, an iris, an anterior capsule, a posterior capsule, or an iridocorneal angle.

16. The system of claim 15, wherein the indication of the location of the at least one RLF includes a semantically segmented RLF.

17. The system of claim 13, wherein to determine the at least one RLF meets at least one detection criteria comprises instructions that, when executed by the one or more processors, cause the system to:
determine one or more phases of the ophthalmic surgical procedure based upon analyzing the surgical image data; and
determine the at least one RLF is detected during a particular phase of the one or more phases of the ophthalmic surgical procedure,
wherein the at least one detection criteria includes detecting the at least one RLF during the particular phase of the ophthalmic surgical procedure.

18. The system of claim 17, wherein a second model determines the one or more phases of the ophthalmic surgical procedure based upon analyzing the surgical image data.

19. The system of claim 13, wherein to detect the at least one RLF within the eye of the subject by the machine learning model includes:
detecting a size of the at least one RLF,
wherein the output data indicates the size of the at least one RLF.

20. The system of claim 13, wherein to detect the at least one RLF within the eye of the subject by the machine learning model includes:
detecting a layer of a lens of the eye of the subject associated with the at least one RLF,
wherein the output data indicates the layer of the lens associated with the at least one RLF.

21. The system of claim 13, wherein to generate the output data comprises instructions that, when executed by the one or more processors, cause the system to:
generate composite surgical image data by generating an overlay on one or more images of the plurality of images of the surgical image data,
wherein the overlay is associated with visually identifying the at least one RLF that meets at least one detection criteria in the one or more images.

22. A non-transitory computer readable storage medium storing computer-executable instructions that, when executed by one or more processors, cause the one or more processors to at least:
obtain surgical image data comprising a plurality of images capturing an ophthalmic surgical procedure of a subject;
provide the surgical image data to a machine learning model trained to detect at least one residual lens fragment (RLF) within an eye of the subject during the ophthalmic surgical procedure based upon analyzing the surgical image data;
determine whether the at least one RLF meets at least one detection criteria;
based upon determining the at least one RLF meets the at least one detection criteria, generate output data associated with detecting the at least one RLF in the eye of the subject; and
transmit the output data to a computing device.

23. The method of claim 1, wherein the preprocessing comprises extracting a region of interest corresponding to the eye and resizing or enhancing the region of interest prior to analysis by the machine learning model.

24. The computer-implemented method of claim 1, wherein detecting the at least one RLF comprises analyzing temporal information across a sequence of images, including determining persistence or motion of the residual lens fragment across multiple frames.
